(19) United States
(12) Patent Application Publication
(10) Pub. No.: US 2022/0000426 A1
(43) Pub. Date: Jan. 6, 2022

(54) MULTI-MODAL BRAIN-COMPUTER INTERFACE BASED SYSTEM AND METHOD

(71) Applicants: Jason FRIEDMAN, Rehovot (IL); Konstantin SONKIN, Ramat Gan (IL)

(72) Inventors: Jason FRIEDMAN, Rehovot (IL); Konstantin SONKIN, Ramat Gan (IL)

(21) Appl. No.: 17/291,476

(22) PCT Filed: Nov. 6, 2019
(86) PCT No.: PCT/IL2019/051211

§ 371 Date: May 5, 2021

Related U.S. Application Data
(60) Provisional application No. 62/756,156, filed on Nov. 6, 2018.

(51) Int. Cl. A61B 5/00; A61B 5/372; A61B 5/384; A61B 5/375; A61N 1/36; G06F 3/01

(52) U.S. Cl. CPC A61B 5/7264; A61B 5/372; A61B 5/384; G06F 3/015; A61B 5/7221; A61B 5/7228; A61N 1/36003; A61B 5/375

(57) ABSTRACT

A multi-modal monitoring system is provided for monitoring activity of an individual. The monitoring system is configured as a computer system comprising data input, memory and a data processor. The data processor is configured and operable to receive and analyze first and second measured data concurrently collected from the individual and corresponding to, respectively, detected brain signals indicative of movement planning by the individual, and detected motion signals indicative of actual movement recognition by at least one body portion of the individual. The data analysis includes applying a multi-modal processing to the first and second measured data to decode the brain and body signals, and upon identifying that the decoded brain and body signals satisfy a condition of common decoded motor commands, generate a control signal indicative of the individual's intended physical action, which can be used for controlling operation of an execution device or assistance device(s), or for providing biofeedback.

### TECHNOLOGICAL FIELD AND BACKGROUND

The present invention is in the field of brain-computer interface (BCI) techniques, and relates to a system and method utilizing non-invasive multi-modal BCI, particularly useful to satisfy the needs of people with movement disabilities.

BCI represents a vast field of potential applications in quality-of-life improvement for patients with severe movement disorders, such as after stroke (Ang, K. K., et al. (2011), Large Clinical Study on the Ability of Stroke Patients in Using EEG-Based Motor Imagery Brain-Computer Interface. Clinical EEG and Neuroscience, 42 (4), 253-258), with lost limbs, paralysis, or with amyotrophic lateral sclerosis (Chaudhary, U., et al., (2015), Brain-Machine Interface (BMI) in paralysis. Annals of Physical and Rehabilitation Medicine, 58 (1), 9-13). BCIs provide a direct communication between a subject's (human) brain and an external device/system. BCI enables direct use of electrical signatures characterizing brain's activity, e.g. for responding to external effects/stimuli. Such interfaces enable subjects to communicate and control devices with commands decoded from brain signals, without using body movements.

There are several approaches for non-invasive acquisition of brain signals for the purposes of BCI, including electrocorticographical signals (Schalk, G., & Leuthardt, E. C. (2011), Brain-computer interfaces using electrocorticographic signals. IEEE reviews in biomedical engineering, 4, 140-154), electroencephalography (EEG) (Ang, K. K., et al., 2011)), magnetoencephalography (Hajipour Sardouie, S., & Shamsollahi, M. B. (2012), Selection of efficient features for discrimination of hand movements from MEG using a BCI competition IV data set. Frontiers in neuroscience, 6, 42), functional magnetic resonance imaging and near-infrared spectroscopy (LaConte, S. M. (2011), Decoding fMRI brain states in real-time. Neuroimage, 56(2), 440-454) and Functional Near-Infrared Spectroscopy (fNIRS) (Naseer, N., & Hong, K. S. (2015). fNIRS-based brain-computer interfaces: a review. Frontiers in human neuroscience, 9, 3). Additionally, in case of partial loss of motor function, as is the case in most motor disorders, eye movements and residual body movements recorded with IMUs (Inertial Measurement Units) [Pierella, C., Abdollahi, F., Thorp, E., Farshchiansadegh, A., Pedersen, J., Seáñez-Gonzalez, I., . . . & Casadio, M. (2017), Learning new movements after paralysis: Results from a home-based study. Scientific reports, 7(1), 4779] can be used instead of brain signals or as an adjunct method. Among these methods, EEG and IMUs are the most affordable, portable and available solutions.

### GENERAL DESCRIPTION

There is a need in the art for a novel non-invasive approach of multi-modal BCI techniques, providing effective decoding of brain and body signals.

The present invention provides a novel technique for monitoring activity of an individual to identify the individual's intended physical action. This can be used for controlling operation of an execution device, and/or assistance device(s) for example to provide feedback to the individual.

As indicated above, the technique of the present invention is intended for quality of life improvement of people with severe movement disorders. There are millions of such people with movement disabilities but modern medical and technical solutions have very limited capability to restore or replace lost motor skills. There is a need for a technology to satisfy the needs of people with movement disabilities to be independent and able to participate in modern life by means of a software-hardware system for touchless control of devices and motor rehabilitation.

One of the known promising, inexpensive and non-invasive approaches is an EEG-based BCI using movement imagery. Currently, implemented interfaces use imaginary movements of large body parts—arms, legs—for classification and control (Doud, A. J., et al., Continuous three-dimensional control of a virtual helicopter using a motor imagery based brain-computer interface, PLoS One, 2011, 6, 10; Wolpaw, J. R. and Wolpaw, E. W., Brain-Computer Interfaces: Principles and Practice, New York: Oxford Univ. Press, 2012), but they have a small repertoire of control commands and a significant time delay. However, development of non-invasive BCIs based solely on EEG signals is complicated because the received signal is inherently weak, noisy and distorted during passage through the brain membranes and skull.

When an individual can still make residual movements, they can be extracted for operating external devices using a Body-Machine Interface (BoMI) [Mussa-Ivaldi, F. A., Casadio, M., & Ranganathan, R. (2013), The body-machine interface: a pathway for rehabilitation and assistance in people with movement disorders. Expert review of medical devices, 10(2), 145-147]. These signals may be extracted directly from body motions, for example using IMU sensors (a combination of accelerometers, gyroscopes and magnetometers) attached to the body, which measure the sensor's acceleration, angular velocity and orientation. The approach is affordable, portable, has high temporal resolution, and can be personalized to the needs of the individual, by selecting which body part to use to control the device, which can vary with time and disease stages. However, IMU based solutions face the problem of accurately detecting voluntary movements as opposed to passive movements (e.g. someone pushing the wheelchair), tremor, or reactive movements (Guerrero-Castellanos, J. F., et al., (2013), A robust nonlinear observer for real-time attitude estimation using low-cost MEMS inertial sensors. Sensors, 13(11), 15138-15158). Additionally, to be relevant for control of devices or motor rehabilitation, the patient needs to be able to produce an appropriate range of movements.

The present invention provides a multi-modal real-time BCI technique for control of devices that overcomes the individual limitations of both EEG-based BCIs and BoMIs. This technique combines brain signals of motor imagery (e.g. registered as EEG) and movement recordings (e.g. by means of IMU sensors such as accelerometers and gyroscopes) using advanced Artificial Intelligence (AI) methods.

In other words, the invention is based on the analysis of combined measured data (concurrently collected/measured from the individual) including brain signals (indicative of movement planning by the individual) and motion signals corresponding to movement recordings (i.e. actual movements recognized from the individual's body portion(s)). The control system of the present invention performs decoding of these brain and body signals by means of AI techniques.

The inventors have demonstrated the fundamental possibility to decode voluntary movements from a combination of residual movement recordings (via accelerometers and gyroscopes) and motor imagery (registered in EEG). The developed AI-based classification system of motor commands performs a cycle of multi-modal multi-channel data acquisition, feature extraction, classification and issuing control commands for software applications or executive devices (such as robotic devices, assistive devices, smartphones and other).

More specifically, the approach of the present invention utilizes parallel acquisition of brain signals/readings (EEG) and motion signals (IMU signals), principal component based feature extraction, spectrum analysis, wavelet transform, time series analysis, and decoding of motor commands by means of classifiers based on machine learning. The technique of the present provides a simple and effective solution for people with severe movement disorders, enabling independence in their work and leisure, via interface for control of smart devices, due to integration of EEG and movement recordings. In contrast to eye-trackers or gesture recognition, the technique of the present invention does not require constant pose or attention.

Thus, according to a broad aspect of the invention, it provides a monitoring system for monitoring activity of an individual. The monitoring system is configured as a computer system comprising data input, memory and a processor. The processor is configured and operable to receive and analyze first and second measured data concurrently collected from the individual and corresponding to, respectively, detected brain signals indicative of movement planning by the individual, and detected motion signals indicative of actual movement by at least one body portion of the individual, and apply a multi-modal processing to the first and second measured data to decode the brain and body signals, and upon identifying that the decoded brain and body signals satisfy a condition of common decoded motor commands, generate a control signal indicative of the individual's intended physical action.

Preferably, the first measured data is indicative of multiple channels of the brain signals originated at multiple sources (and locations) distributed in the brain. The processor is thus configured and operable to determine frequency and time evolution of the brain signals corresponding to the multiple sources distributed in the brain.

The processor is configured and operable to determine a time pattern of the motion signal being indicative of a motion type and quality. Preferably, the second measured data is indicative of the motion signals originating at two or more different locations on the body portion of the individual.

The processor is preprogrammed to apply machine learning analysis to the brain signals and to the motion signals to define, for each of these signals, an optimal set of features to be identified in the respective measured data.

More specifically, the processor includes a data analyzer system and a validation utility. The data analyzer system includes: a first data analyzer configured and operable to apply model-based analysis to the first measured data and identify in the detected brain signals a first set of features characterizing classified brain-related motor commands; and a second data analyzer configured and operable to apply a model-based analysis to the second measured data and identify in the detected motion signals a second set of features characterizing one or more classified movements. The validation utility is connected to the first and second analyzers and is configured and operable to determine whether data indicative of the first and second sets of features satisfy the condition of common decoded motor commands corresponding to the individual's intended physical action resulting in the one or more movements.

The first analyzer is preferably configured and operable to apply machine learning analysis to the brain signals to define said first set of features characterizing the classified brain-related motor commands. Similarly, the second analyzer is preferably configured and operable to apply machine learning analysis to the motion signals to define said second set of features characterizing one or more classified movement

The monitoring system may also include an operating utility configured and operable to analyze the control signal indicative of the individual's intended physical action to select a corresponding physical action to be performed by an execution device.

In some embodiments, the monitoring system may include a communication utility connected to the processor and configured and operable to analyze output data provided by the processor and generate feedback data indicative of whether said condition is satisfied or not to be communicated to the individual.

Thus, the data analyzer system is configured to define the first and second data analysis channels for analyzing the first and second measured data, respectively. The first data analyzer comprises a first feature extractor utility configured and operable to extract from the first measured data a plurality of features associated with motor commands, and a first classifier utility configured and operable to utilize machine learning results to assign classification data to the first set of features from said first plurality of features, and generate corresponding first classification data associated with the classified brain-related motor commands. The second data analyzer comprises a second feature extractor utility configured and operable to extract from the second measured data a second plurality of features associated describing one or more movements, and a second classifier utility configured and operable to utilize machine learning results to assign classification data to the second set of features from said second plurality of features, and generate corresponding second classification data characterizing the one or more classified movements. By this, first and second classification data are provided associated with, respectively, the first and second measured data. The validation utility operates to determine whether the first and second classification data satisfy a condition of mutual validation of the motor command decoding obtained from the first and second measured data.

The extractor utility is configured to utilize one or more predetermined models and apply a model-based processing to the respective measured data and extract the set of features associated with/indicative of the motor commands Each of the first and second measured data includes a pattern of measured signals, and the extractor utility analyzes the respective pattern to identify and extract one or more features characterizing an individual's intended physical action. Such a pattern corresponds to a movement signature being measured, and may be a multi-parameter function, e.g. the function of time, frequency and spatial signal distributions.

For example, for the actual motion signals pattern, such optimal features may include kinematic landmarks (e.g. velocity or acceleration peaks). The second extractor utility may thus be configured and operable to analyze the at least one motion signals relating pattern and extract at least kinematic landmarks to be included in the second set of features. As for the brain signals, such optimal features may include descriptive features specific to imaginary movements of the body portion from which the actual motion signals are being collected.

The monitoring system may include a preliminary analyzer configured and operable to analyze the first measured data indicative of the brain signals, and upon identifying movement related signals in the first measured data, utilizing said movement related signals as a marker of voluntary movement onset to select for analysis a part of the second measured data being collected from said voluntary movement onset.

The monitoring system may be configured and operable for data communication with a measured data provider to receive therefrom said first and second measured data, and configured and operable for signal communication with at least one of an execution device and an individual's assistant device to communicate data indicative of the individual's intended physical action. The measured data provider may be a storage system where the first and second measured data are stored.

Thus, the monitoring system of the present invention may be a stand alone system connectable to the measured data provider, which in turn is in communication with first and second measurement devices to receive therefrom said first and second measured data indicative of concurrently measured brain and motion signals.

Important aspect of the monitoring system, being stand alone or integrated system, is its capability to perform real time analysis and decoding of activity of an individual to identify the individual's intended physical action. This advantageously allows the system to be used for controlling operation of an execution device or assistance device(s), and for providing biofeedback.

In some embodiments, the monitoring system, being a computer system, may be part of (installed in) one of the first and second measurement devices and being configured to communicate with the other of the measurement devices to thereby receive both the first and second measured data. In addition, the monitoring system may also include a controller to synchronize concurrent measurements by the first and second measurement devices.

The invention in another broad aspect provides a measurement system for use in monitoring activity of an individual. The measurement system comprises: a first measurement device configured and operable to detect brain signals of an individual and generate first measured data indicative of movement planning by the individual; a second measurement device comprising at least one motion sensor configured for placement on at least one portion of a body of the individual and generate second measured data indicative of actual movement recognition by said at least one body portion; and the above described monitoring system.

The invention also provides a measurement system for use in monitoring activity of an individual, where the measurement system comprises: a first measurement device configured and operable to detect brain signals of an individual and generate first measured data indicative of movement planning by the individual; and the above-described monitoring system configured and operable to receive said first measured data and to communicate with a second measurement device to receive therefrom second measured data indicative of actual movement recognition by at least one body portion being measured concurrently with said brain signals measurements.

The invention also provides a method for use in monitoring activity of an individual. The method comprises: providing first and second measured data collected from an individual and corresponding to, respectively, detected brain signals indicative of movement planning by the individual, and motion signals indicative of actual movement recognition by a body portion of the individual, and processing and analyzing the first and second measured data to generate a control signal indicative of the individual's intended physical action, said processing and analyzing comprising applying a multi-modal processing to the first and second measured data to decode the brain and body signals, and upon identifying that the decoded brain and body signals satisfy a condition of common decoded motor commands, generate a control signal indicative of the individual's intended physical action.

### BRIEF DESCRIPTION OF THE DRAWINGS

In order to better understand the subject matter that is disclosed herein and to exemplify how it may be carried out in practice, embodiments will now be described, by way of non-limiting examples only, with reference to the accompanying drawings, in which:

FIG. 1 is a block diagram of the monitoring system of the invention for monitoring activity of an individual to identify individual's intended physical action;

FIG. 2 schematically illustrated a flow diagram of the implementation of the technique of the present invention for monitoring the activity of an individual;

FIG. 3 schematically illustrates a flow diagram of a specific example of the method of the invention for monitoring the activity of an individual to identify individual's intended physical action and determine a corresponding physical action to be performed by an execution device;

FIGS. 4A-4F exemplify the measured data acquisition and data analysis according to the technique of the present invention, wherein FIGS. 4A and 4B-show the IMU data acquisition and analysis using two IMUs placed on the individual's shoulders; and FIGS. 4C-4F show the EEG data acquisition and analysis using an EEG cap (electrodes' arrangement) and amplifier;

FIG. 5 is block diagram illustrating schematically an exemplary method of implementing the technique of the present invention; and

FIG. 6 is a schematic illustration of the flow diagram of the exemplary monitoring system operation utilizing the machine learning and data analysis according to the invention, implemented using a phone device.

### DETAILED DESCRIPTION OF EMBODIMENTS

As described above, the present invention provides a novel monitoring system and method for monitoring activity of an individual to identify the individual's intended physical action, which in some examples may be used for controlling operation of an execution device. The invention utilizes multi-modal (and preferably real-time) analysis of measured data including data indicative of brain signals, such as EEG, and motion data (movement recordings, such as IMU signals) detected concurrently with the detection of at least part of the brain signals, where the measured data analysis utilizes advanced AI techniques.

In some embodiments, the invention is aimed at touchless control of various devices (i.e. performing/initiating a physical effect on the device), for an individual with severe movement disorders, who would otherwise not be able to perform such effect; and for motor training for such an individual. The technique of the present invention can use relatively weak non-invasively measured brain-reading signals (EEG or the like signals).

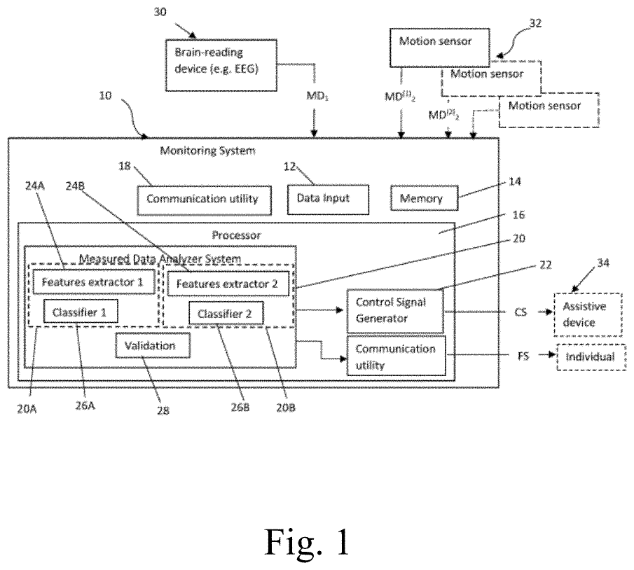

FIG. 1 illustrates, by way of a block diagram, the main principles of the technique of the present invention. The invention provides a monitoring system 10, which is configured generally as a computer system, including inter alia data input utility 12, memory 14, processor 16, data output (e.g. including display). The system 10 may also include an appropriate communication utility 18 for data/signal communication with external devices via wires and/or wireless signal communication using any known suitable techniques/protocols. Such external devices may include a measured data provider, which may be constituted by a storage utility, being either a separate storage where the previously collected measured data is stored for off-line mode analysis or memory/storage of measurement systems enabling real time data analysis. Further the same or another communication utility may be used for communicating output data indicative of the analysis results to the individual's assistant device and/or an execution device, as will be described further below. Thus, the monitoring system may receive measured data directly from the measurement device(s) to perform on-line data analysis or from a storage device to perform off-line data analysis. In any case, the measured data being analyzed includes brain signals and motion signals which are concurrently collected/measured from the individual by the respective measurement devices. It should be noted that the monitoring system may receive measured data of two types—brain signals and motion signals, while the number of the measurement devices providing data of each type can be more than one, and the type of the devices is not limited by a specific type of motion sensors or sensors for brain signal acquisition. The system may receive data as a combination of different brain sensors and motion sensors.

As exemplified in the figure, the monitoring system 10 is configured and operable to communicate with a measured data provider, which in the present not limiting example is constituted by measurement devices, including one or more brain monitoring devices 30 (e.g. EEG) and one or more motion sensing devices, generally at 32, and to communicate with one or more assistive devices, generally at 34. As described above, the monitoring system 10 may be a stand alone system configured for data communication with the measured data provider, or may be a part of one of the measurement devices 30 and 32. Generally, the data processing utilities of the monitoring system may be distributed in both measurement devices, as the case may be.

The monitoring system 10 receives measured data MD₁ from the brain reading/monitoring device 30 and measured data MD₂ (or multiple such measured data pieces MD⁽¹⁾₂, MD⁽¹⁾₂, etc.) from motion sensor(s) 32 which operated concurrently with the brain reading device 30 to collect the motion data concurrently with the brain signals. The construction and operation of such measurement devices do not form part of the present invention and any known suitable measurement devices of the type specific can be used, and therefore these devices need not be described in detail.

For the purposes of the invention, the measured data MD₁ (e.g. EEG data) corresponds to detected brain signals and is indicative of individual's brain activity, while the individual “intends” to perform some physical activity, which is sensed by motion sensor(s) generating corresponding measured data MD₂.

Typically, the first measured data MD₁ is indicative of multiple channels of the brain signals originated at multiple sources distributed in the brain. This may be actually a location map of the detected signals. Such measurements may be performed using an array of electrodes (e.g. EEG cap) which might be further assisted by fMRI data. The second measured data is preferably indicative of the motion signals originated at two or more different locations within the body portion of the individual.

The processor 16 is configured and operable to analyze, by parallel model-based processing, the data indicative of the brain signals and the data indicative of the motion signals. The brain signals are indicative of movement planning by the individual, and the motion signals are indicative of actual movement recognition by the body portion of the individual. The multi-modal processing of the first and second measured data is aimed at decoding the brain and body signals, and upon identifying that the decoded brain and body signals satisfy a condition of common decoded motor commands, generate a control signal indicative of the individual's intended physical action. As will be described more specifically further below, the processor is preferably configured and operable to analyze the first measured data MD₁ to determine frequency and time evolution of the brain signals corresponding to the multiple sources distributed in the brain; and is preferably configured and operable to determine a time pattern (signature) of the motion signal which is indicative of a motion type and quality.

The processor 16 includes a measured data analyzer system 20 which includes first and second analyzers 20A and 20B configured and operable to process the two types of measured data MD₁ and MD₂, respectively, and “decides” about the individual's brain activity associated with movement(s) performed by the individual, and generates corresponding decision data. This data is further processed by a control signal generator 22, which generates the control signal in case the individual's brain activity data associated with the detected motion signals is indicative of the individual's intended physical action. In some embodiments, the control signal may be used to operate a respective external device 34 and/or may be used for communicating feedback data/signal FS to the individual, e.g. via the individual's personal communication device, such as a phone device.

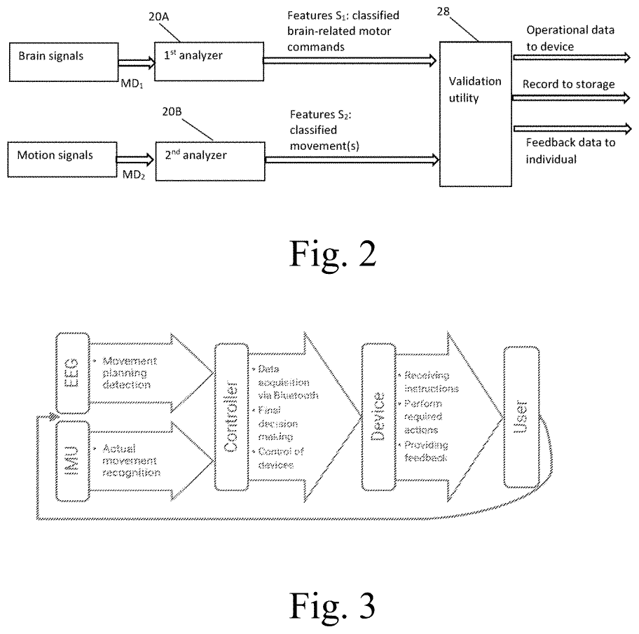

Referring to FIG. 2, there is schematically illustrated a flow diagram of the implementation of an example of the present invention. The brain activity measured data MD₁ and the motion signal measured data MD₂ are concurrently collected from the individual and undergo processing by, respectively, the first and second analyzers 20A and 20B, where the brain activity data MD₁ (brain signals) is indicative of “movement planning” by the individual, while the motion signal data MD₂ (motion signals) is indicative of actual movement recognition. The analyzer 20A is configured and operable to apply model-based analysis to the data MD₁ indicative of the brain signals and identify in the detected brain signals a first set of features S₁ characterizing classified brain-related motor commands. The analyzer 20B is configured and operable to apply a model-based analysis to the measured data MD₂ indicative of the motion signals and identify in the detected motion signals a second set of features S₂ characterizing one or more classified movements. The so-determined classified features' sets S₁ and S₂ undergo validation processing by the validation utility 28, which is configured and operable to determine whether the classified features' sets S₁ and S₂ satisfy the condition of common decoded motor commands corresponding to the individual's intended physical action resulting in the detected one or more movements.

For example, the first analyzer 20A calculates/determines a set of features based on brain signals and classify them as a specific imaginary movement. The second analyzer 20B extracts descriptive features of motion signals, which includes motion signature for classification of a specific movement as well as parameters of the movement quality. Motion signature and descriptive characteristics of movement quality may include kinematic landmarks, velocity and acceleration profiles and number of extremums, movement trajectory, movement duration, number of breakpoints and other.

FIG. 3 shows more specifically an example of the implementation of the technique of the present invention. As described above, the monitoring system 10 receives measured data from the measured data provider, and the measured data includes brain signals and motion signals collected concurrently from the individual by e.g. the EEG brain reading system and the IMU motion sensor system. The monitoring system 10 performs data acquisition via wired and/or wireless (Bluetooth-based) communication utility 18. The processor 16 operates to perform final decision making as described above, and, upon identifying the existence of the above condition of common decoded motor commands in the brain and body motion signals, generate the control signal CS. In some embodiments, the control signal may be used to generate operating instructions. The operating instructions may be used to operate the assistance device 34 to execute the respective action, and/or to generate feedback data to the user (individual). Thus, the control signals generated by the monitoring system actually present decoded motor commands which can be in real time applied to control of assistive devices, robots, smartphones and exoskeletons as well as for biofeedback training with visual feedback.

Thus, turning back to FIG. 1, the processor 16 receives and analyzes the first and second measured data MD₁ and MD₂ of different types concurrently collected from an individual and corresponding to, respectively, detected brain signals indicative of movement planning by the individual and acquired motion signals, which might contain recordings of residual movements of an individual. Each of the first and second measured data is indicative of motor commands. The processor 16 applies a multi-modal processing to the first and second measured data, and upon identifying that the first and second data satisfy a condition of common decoded motor commands, generates a control signal indicative of the individual's intended physical action.

The basic principle of the multi-modal monitoring system 10 is to use mutual validation of motor command decoding, obtained by pattern recognition applied to both the motion signals (e.g. measured by IMUs) and the brain signals (e.g. measured by EEG system). More specifically, the monitoring system 10 (i.e. data analyzer 20) applies independent model-based processing (decoding) to the measured data MD₂ (motion data/signals) and to the measured data MD₁ (brain signals); and to apply mutual validation of such IMU data and the EEG data.

As will be described further below, in some embodiments, the validation is performed as follows. Upon identifying (by a classifier) in one of the measured data MD₁ and MD₂ a certain pattern corresponding to motion command, this is validated from the other of these two types of measured data (using another respective classifier). If one classifier recognizes the pattern corresponding to a motor command, the other classifier has to validate it, with minimal time delays. To this end, machine learning/training is performed to define an optimal set of features to be identified in the EEG and IMU patterns. Such an optimal set of features is a set of features characterizing classified motor commands: the first set of features extracted from the first measured data characterizes classified brain-related motor commands; and the second set of features extracted from the second measured data characterize one or more classified movements.

The data analyzer 20 is configured to define first and second data analysis channels (first and second analyzers 20A and 20B) for performing analysis of the first and second measured data, respectively. Each of the first and second data analysis channels comprises a feature extractor 24A, 24B configured to extract from the respective measured data a plurality of features associated with motor commands.

The extracted plurality of features is then processed by a respective classifier 26A, 26B, which performs machine learning, and, based on the machine learning results, assigns classification data to the respective selected set of features from said plurality of features. The so-produced first and second classification data include first classification data associated with the classified brain-related motor commands and second classification data characterizing the one or more classified movements. These classification data are analyzed by the validation utility 28 which operates to determine whether these classification data satisfies a condition of mutual validation of the motor command decoding obtained from the first and second measured data.

The following is the description of a specific but not limiting example of the use of the present invention for decoding the actual and planned movement data for controlling operation of the assistant device. In this example, small shoulder movements decoding is implemented by recording and analyzing the measured data. Most individuals with severe motor disorders (even after most spinal cord injuries) can still make these residual movements. The inventors have demonstrated the efficiency of the invention when operating in the most common mode.

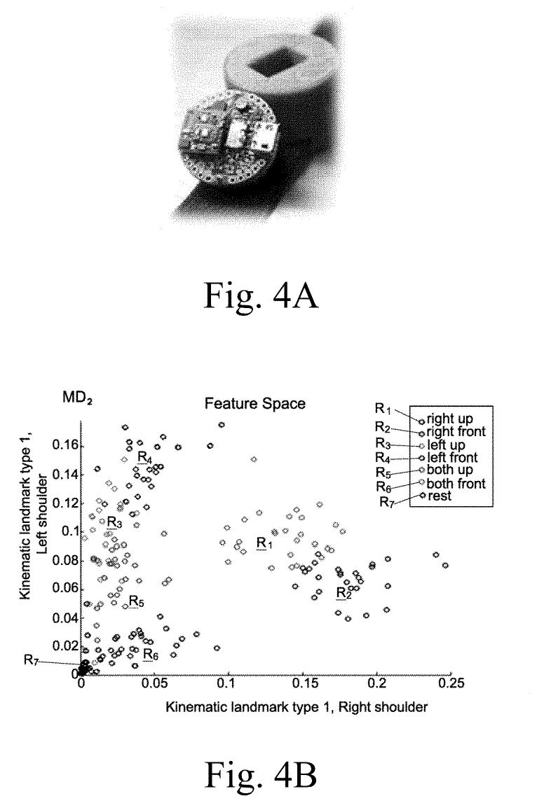

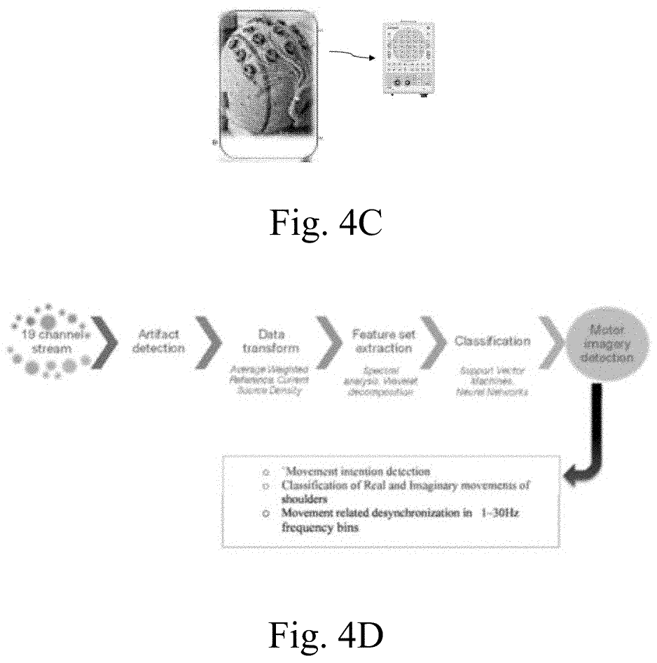

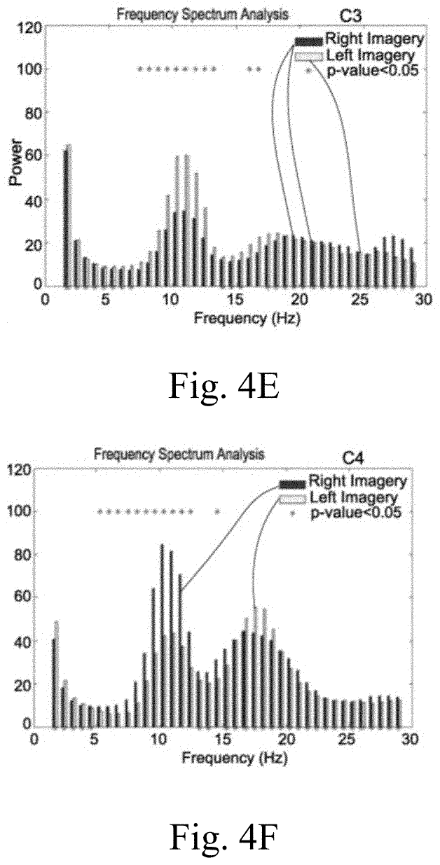

FIGS. 4A and -4B illustrate the motion data acquisition and analysis, which in this example utilize IMU motion data. FIG. 4A shows two IMUs (motion sensors) placed on the individual's shoulders. The IMUs operate to record 6 types of shoulder movements, and rest. As shown in FIG. 4B, the so-collected motion data MD₂ was processed by applying thereto principal component analysis (PCA), which was performed on the 14-dimensional data to decrease its dimensionality to the two dimensions. Kinematic landmarks (e.g. velocity and acceleration extremums) were used in the plurality of features and were extracted from the principal components for each IMU. As an example, a scatter plot of one type of features—kinematic landmarks in the form of amplitude of velocity peaks extracted from the first principle component of motion data from two sensors placed on shoulders—is shown in FIG. 4B. In the graph, the x-axis represents the magnitude of the velocity profile extremum registered by the sensor placed on the right shoulder, and the y-axis represents the magnitude of the velocity profile extremum registered by the sensor placed on the left shoulder. Here, 7 different movement states can be observed for the right and left shoulders—right up, right front, left up, left front, both up, both front, and rest, the respective measured signals being distributed in respective parametric space regions R₁-R₇. A classifier, based on machine learning, achieved classification accuracy for 7 movements 80±11% and for 4 movements—95%±5% on average (5-fold cross-validation was performed), and the classified set of features was generated. This set of features included velocity and acceleration profiles, magnitude of its peaks, number of its extremums, movement trajectory, active movement duration and number of breakpoints in the velocity and acceleration profiles.

FIGS. 4C-4F illustrate the EEG data acquisition and analysis. FIG. 4C illustrates an EEG cap (electrodes' arrangement) and amplifier used for detection of the EEG signals. As shown in FIG. 4D, the so-collected EEG data MD₁, corresponding to imaginary right and left shoulder movements was recorded and analyzed. In this example, this data includes 19 channel EEG data (sampling rate 500 Hz). After artifact detection and data preprocessing steps, EEG data was transformed to the Power-Frequency domain and divided to narrow bands (0.71 Hz). All bands were tested for difference under two labels—imaginary left or right shoulder movement with two-sided t-tests for independent samples with α=0.05. Descriptive features specific to the imaginary movements of right and left limbs were extracted, i.e. frequency bands where the power showed significant differences between conditions, which is illustrated in FIGS. 4E and 4F, showing movement related desynchronization in 1-30 Hz frequency bins in different EEG channels (95 trials were averaged over labels; trial length of 1.4 sec).

As described above, the target of the EEG signal analysis is to detect movement intention (i.e. individual's intended physical action). Depending on actual movement capabilities of an individual, it is used in two regimes. First, in case residual movements are available for a subject (individual), the EEG movement detection is used as a marker of voluntary movement onset, as opposed to tremor or passive movement. In this case, the number of control commands varies from 3 to 7 with classification accuracy from 80 to 95 percent on average correspondingly. Second, in case of a locked-in subject (no residual movements are available), the EEG decoding itself might be used for control of assistant device(s). In this case, the amount of control commands varies from 2 to 4 and accuracy is lower than in multi-modal regime.

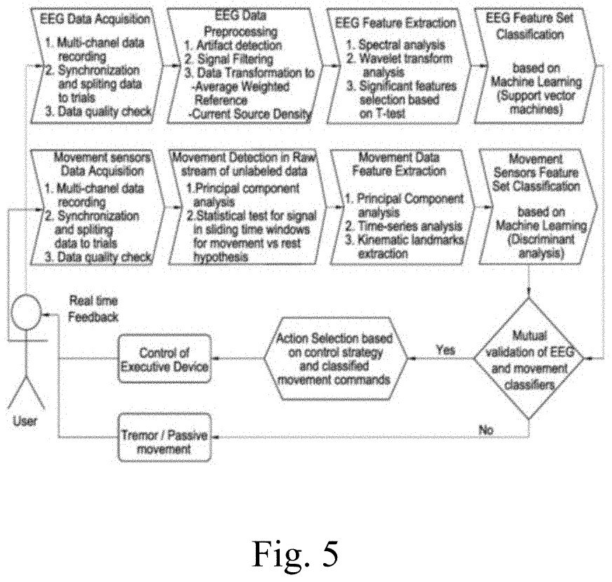

FIG. 5 illustrates, by way of the block diagram, a specific but not limiting example of the above-described multi-modal BCI technique of the present invention. As shown, the EEG data acquisition and the motion data acquisition are concurrently and independently performed providing the movement planning and actual movement measured data MD₁ and MD₂.

The initiation of any muscle's movement can be seen in unique electric patterns contained in EEG. Similar patterns can also appear even when muscles do not contract—by imagined movements or in cases such as paralysis or amputation. Hence, the first objective of the current multi-modal BCI system is real-time analysis of EEG allowing prediction of the intended movement, based on EEG data.

The second objective is real-time analysis of movement recordings as most of the target users can still make small residual movements, e.g. movements of shoulders. These residual movements can serve as the first targets for intervention. The structure of the multi-modal BCI is based on parallel acquisition and decoding of EEG, for neural patterns data, and IMU signals for actual limbs movement recording. The system performs, in parallel, the following main steps: (1) real time acquisition of EEG and IMU signals; (2) signal preprocessing; (3) advanced feature extraction, including principal component analysis, spectrum analysis and wavelet transform; (4) automatic feature selection; (5) decoding of motor commands by means of classifiers based on machine learning; and (6) control of devices or providing feedback to the user.

Each of two types of data (MD₁ and MD₂) acquisition sessions includes multi-channel data recording, and pre-processing of the detected signals, including synchronization and splitting data to trials and data quality analysis. Synchronization of the corresponding trials is performed using internal time stamps and specific cues presented during data acquisition in several data streams Data quality analysis includes verification that acquired data contains unseen data samples with unique time stamps. Then, the measured data MD₁ and MD₂ undergo data analysis in two parallel independent (separate) processing channels.

The EEG data MD₁ is processed, as described above, to detect the artifacts (high-amplitude noise related to non-relevant activity, e g blinks and eye-movements), apply signal filtering, and data transformation. Signal filtering is performed to filter out noise induced by the electrical network and other sources of electromagnetic fields. Combination of several filters is used for brain data: low pass filters (up to 30 Hz), high-pass filters (from 0.5 Hz) and notch filter (40-60 Hz). Data transformation step includes transform of EEG data to either weighted average reference or current source density. Weighted average reference montage and current source density transforms produce a spatial filtering effect and diminish the influence of a common reference in the EEG signal. These approaches aim to diminish the factors that cause an interdependence of EEG signals and lower volume conductance effects from neighboring areas in the brain. After artifact detection and data preprocessing steps, descriptive characteristics of EEG data related to movement imagery are extracted using several approaches—spectral analysis and wavelet transform.

In spectral analysis step EEG data is transformed to the Power-Frequency domain and divided to narrow bands (0.7-2.0 Hz). This step is followed by feature selection, when all bands are tested for difference under labels corresponding to imaginary movements (e.g. imaginary movements of shoulders) with two-sided t-tests for independent samples with α=0.05. Descriptive features specific to different imaginary movements are extracted, i.e. frequency bands where the power showed significant differences between conditions.

In alternative method for feature extraction based on wavelet transform EEG data is decomposed into a linear combination of basis functions, which are dilated versions of a single function—the mother wavelet function. Since the wavelet functions are finite in time this type of analysis obtains information about time localization of features in addition to frequency data, which is important since the patterns of the brain activity are related to time variations of EEG signal. In this example, discrete wavelet transform is applied using Daubechies and Symlet mother wavelet families and the level of decomposition that will provide accurate frequency ranges corresponding to the bands of interest between 5 and 30 Hz. Extracted features are the coefficients received from the decomposition of the EEG signal using the described discrete wavelet transform. These features form a unique representation of the signal, characterized by the mother wavelet family and the level of decomposition.

At the next step extracted features are used to train a Support Vector Machine classifier. Once a classifier was trained, new unlabeled EEG segments could be classified by it in real-time

In parallel, motion related data MD₂ is processed and analyzed. The processing is applied to raw data stream. At the first step, a movement onset is detected in a stream of data. It is achieved by means of principle components analysis followed by statistical Kolmogorov-Smirnov test for signals in sliding time windows in order to test movement vs rest hypothesis. If the movement onset is detected, feature extraction is performed for movement data as described above, which includes calculation of motion signature and parameters of the movement quality. At the next step movement feature classification based on machine learning is performed. Several classifiers are used, including linear discriminant analysis, support vector machine and artificial neural network.

The results of the EEG feature classification and the movement sensors feature classification are indicative of optimal sets of features identified in the EEG and IMU patterns, and they are further analyzed by mutual validation. If the pattern corresponding to a motor command in the EEG data is validated in the movement pattern (or vice versa), then the process proceeds to the selection of physical action to be performed, according to predetermined control strategy and classified movement commands enabling control of the executive/assistant device; otherwise the measured data is treated/interpreted as tremor/passive movement. The respective feedback is provided to the user.

The system has been tested in several studies and demonstrated following results.

The movement data analysis were performed as follows: For movement data acquisition, two IMU sensors were placed on two shoulders inside special bracelets. Four types of shoulder movements and rest were recorded. The training and testing of the system used the same experimental design: the movement sequence consisted of 20 movements, 5 of each type, in a random order. Ten subjects participated in two types of experiments—“Lab Conditions” and “Day to Day Usage” groups. In “Lab Conditions” experiment participants trained the system and immediately tested it. In “Day to Day Usage” experiment, a previously trained classification model was tested. In the movement data analysis, the “Lab Conditions” experiment showed a mean classification accuracy for actual shoulder movement of 98±2%, whereas in “Day to Day Usage” the mean accuracy was 89±3%. The main reason for decline of average accuracy in the “Day to Day Usage” experiment was likely changes in location of the sensors and in the posture of the participant, which were significantly different in training and testing. As chance level for 4 classes classification is, on average, 25%, both results prove to be significantly accurate.

The EEG data analysis were performed as follows: EEG was recorded during the “training” protocol, where subjects moved (or imagined movement) at cue. Nine subjects completed 27 minutes training protocol, instructing subjects to move one or two shoulders at cue or imagine the movement. The sequence was random for the side of movement (Left/Right/Both). For each side, the first three cues were for real shoulder movements and the last three cues were for imagined movement of the same side. EEG data was collected using 19 head electrodes and 2 ear-reference electrodes. In EEG classification, parameter adjustment had led, in some subjects, to 90% accuracy of classification (by training on 80% of the data and testing on 20% unseen trials). Total results indicate moderately good accuracy level significantly higher than chance.

The above results of the multi-modal BCI demonstrate the fundamental possibility to decode intended movements from residual movement recordings and from motor imagery (even in case of absence of any motor activity) in real time.

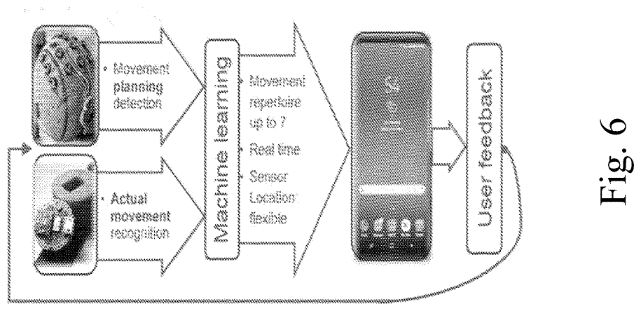

As described above, the present invention utilizes machine learning applied to the EEG and IMU data to define the optimal sets of features characterizing patterns having common “meaning”. FIG. 6 schematically illustrates the flow diagram of the monitoring system operation utilizing the machine learning and data analysis. In this specific not limiting example, the assistant device 34 is a smartphone device, which is configured to be responsive to control signals from the control system of the present invention to perform the required action (i.e. actuate one or more of the phone utilities) and to generate feedback data. Practically, the control system of the invention may be part of/installed in such phone device.

Thus, the novel technique of the present invention utilizes the combined multi-modal data, i.e., the combination of brain signals recorded using any suitable known technique (e.g. by EEG) and preferably from multiple locations/channels within the brain region, and corresponding movement recordings using any known suitable motion sensor (e.g. via IMUs) preferably from more than one location on the individual's body. This in turn allows the mutual validation of motor command decoding obtained from both IMU and EEG pattern recognition, which allows to overcome tremor, passive movements and provide touchless control of various execution/assistant devices for users with wide range of movement disabilities—from light to severe movement disorders.

Also, it should be noted that the inventors have achieved recognition of real and imaginary shoulder movements from the brain readings (EEG) data, which is a complicated task because of the very small representation of neurons relevant for control of shoulder movements in motor cortex in comparison with hand or wrist movements. The invention provides for movement onset detection and classification from raw brain and motion data. The control system of the present invention can perform real time acquisition, analysis and decoding of multi-modal data, which is crucial for its ability to be used in control of devices and as biofeedback training for rehabilitation purposes.

The invention can be used for touchless control of devices such as personal computer (laptop) and smartphone in special accessibility mode. The invention can be used for communication and independent control of applications using only imaginary movements and actual residual movements available for a user. Automatic adaptation is available for users with different motor capabilities—from light impairment of fine motor skills (caused by stroke, TBI, etc.) to severe disabilities and even paralysis (e.g. caused by spinal cord injuries).

The invention can also be used for motor training for patients with severe movement disorders combining motor imagination and corresponding actual movement attempts. Such a system facilitates both restoration of impaired brain function and elaboration of residual movements, based on feedback from the combination of relevant brain and movement signals. In addition, the composition of descriptive features of brain and body data contains important information for monitoring the dynamics of motor learning in rehabilitation period. It allows the monitoring system to combine information about how the brain plans the movement and what is the corresponding motor outcome of such planning. For instance, positive dynamics of data analysis and classification in one data modality and lack of dynamics in another data modality can indicate aspects of rehabilitation that hinder recovery.

By relying on brain plasticity, multi-modal BCI helps train persisting cortical connections to execute motor output of the motor-impaired limb (e.g., hand). The BCI-based technique of the invention can advantageously be used as an assistive solution to traditional physiotherapeutic approaches; and the BCI-based biofeedback system can be used for motor post-stroke recovery.

1-36. (canceled)

37. A monitoring system for monitoring activity of an individual; the monitoring system being configured as a computer system comprising data input, memory and a processor, the processor being configured and operable to receive and analyze first and second measured data concurrently collected from the individual and corresponding to, respectively, detected brain signals indicative of movement planning by the individual, and detected motion signals indicative of actual movement recognition by at least one body portion of the individual, and apply a multi-modal processing to the first and second measured data to decode the brain and body signals, and upon identifying that the decoded brain and body signals satisfy a condition of common decoded motor commands, generate a control signal indicative of the individual's intended physical action.

38. The monitoring system according to claim 37, wherein at least one of the following is true:
a. said first measured data is indicative of multiple channels of the brain signals originated at multiple sources distributed in the brain;
b. said second measured data is indicative of the motion signals originated at two or more different locations within said at least body portion of the individual;
c. processor comprises:
a first data analyzer configured and operable to apply model-based analysis to the first measured data and identify in the detected brain signals a first set of features characterizing classified brain-related motor commands;
a second data analyzer configured and operable to apply a model-based analysis to the second measured data and identify in the detected motion signals a second set of features characterizing one or more classified movements; and
a validation utility connected to the first and second analyzers and being configured and operable to determine whether data indicative of the first and second sets of features satisfy the condition of common decoded motor commands corresponding to the individual's intended physical action resulting in the one or more movements;
d. the monitoring system comprises an operating utility configured and operable to analyze the control signal indicative of the individual's intended physical action to select a corresponding physical action to be performed by an execution device;
e. the monitoring system comprises a communication utility connected to the processor and configured and operable to analyze output data provided by the processor and generate feedback data indicative of whether said condition is satisfied or not to be communicated to the individual;
f. the detected brain signals comprise EEG signals;
g. the monitoring system comprises a preliminary analyzer configured and operable to analyze the first measured data indicative of the brain signals, and upon identifying movement related signals in the first measured data, utilizing said movement related signals as a marker of voluntary movement onset to select for analysis a part of the second measured data being collected from said voluntary movement onset;
h. the monitoring system is configured and operable for data communication with a measured data provider to receive therefrom said first and second measured data, and configured and operable for signal communication with at least one of an execution device and an individual's assistant device to communicate data indicative of the individual's intended physical action.

39. The monitoring system according to claim 38, wherein the processor is configured and operable to determine frequency and time evolution of the brain signals corresponding to the multiple sources distributed in the brain.

40. The monitoring system according to claim 39, wherein the processor is configured and operable to determine a time pattern of the motion signal being indicative of a motion type and quality.

41. The monitoring system according to claim 38, wherein the first analyzer is configured and operable to apply machine learning analysis to, the brain signals to define said first set of features characterizing the classified brain-related motor commands; and the second analyzer is configured and operable to apply machine learning analysis to the motion signals to define said second set of features characterizing one or more classified movements.

42. The monitoring system according to claim 41, wherein:
the first data analyzer comprises a first feature extractor utility configured and operable to extract from the first measured data a first plurality of features associated with motor commands, and a first classifier utility configured and operable to utilize machine learning results to assign classification data to the first set of features from said first plurality of features, and generate corresponding first classification data associated with the classified brain-related motor commands;
the second data analyzer comprises a second feature extractor utility configured and operable to extract from the second measured data a second plurality of features associated describing one or more movements, and a second classifier utility configured and operable to utilize machine learning results to assign classification data to the second set of features from said second plurality of features, and generate corresponding second classification data characterizing the one or more classified movements; and
said validation utility is configured and operable to determine whether the first and second classification data satisfy a condition of mutual validation of the motor command decoding obtained from the first and second measured data.

43. The monitoring system according to claim 42, wherein the second measured data comprises at least one time pattern of the motion signals sensed on at least one portion of the body, and the second extractor utility being configured and operable to analyze said at least one time pattern and extract at least kinematic landmarks to be included in the second set of features.

44. The monitoring system according to claim 42, wherein the first extractor utility is configured and operable to extract from the first measured data descriptive features specific to imaginary movements of said at least one portion of the body to be included in the first set of features.

45. The monitoring system according to claim 38, wherein the measured data provider comprises a storage system where the first and second measured data are stored.

46. A measurement system for use in monitoring activity of an individual, the measurement system comprising:
at least one first measurement device configured and operable to detect brain signals of the individual and generate first measured data indicative of movement planning by the individual;
at least one second measurement device comprising at least one motion sensor configured for placement on at least one portion of a body of the individual and generate second measured data indicative of actual movement recognition by said at least one body portion; and
a monitoring system for monitoring activity of an individual, the monitoring system being configured as a computer system comprising data input, memory and a processor, the processor being configured and operable to receive and analyze first and second measured data concurrently collected from the individual and corresponding to, respectively, detected brain signals indicative of movement planning by the individual, and detected motion signals indicative of actual movement recognition by at least one body portion of the individual, and apply a multi-modal processing to the first and second measured data to decode the brain and body signals, and upon identifying that the decoded brain and body signals satisfy a condition of common decoded motor commands, generate a control signal indicative of the individual's intended physical action.

47. The measurement system according to claim 46, wherein at least one of the following is true:
a. the first and second measured data are concurrently collected; and
b. the control system is configured and operable to analyze the first measured data, and upon identifying movement related signals in the first measured data, utilizing said movement related signals as a marker of voluntary movement onset, to initiate recording and analysis of the second measured data.

48. A method for use in monitoring activity of an individual, the method comprising:
providing first and second measured data collected from an individual and corresponding to, respectively, detected brain signals indicative of movement planning by the individual, and motion signals indicative of actual movement recognition by a body portion of the individual, and
processing and analyzing the first and second measured data to generate a control signal indicative of the individual's intended physical action, said processing and analyzing comprising applying a multi-modal processing to the first and second measured data to decode the brain and body signals, and upon identifying that the decoded brain and body signals satisfy a condition of common decoded motor commands, generate a control signal indicative of the individual's intended physical action.

49. The method according to claim 48, wherein at least one of the following is true:
a. said first measured data is indicative of multiple channels of the brain signals originated at multiple sources distributed in the brain;
b. said second measured data is indicative of the motion signals originated at two or more different locations within said at least body portion of the individual;
c. said processing comprises:
applying model-based analysis to the first measured data and identifying in the detected brain signals a first set of features associated with classified brain-related motor commands;
applying a model-based analysis to the second measured data and identifying in the detected motion signals a second set of features characterizing one or more classified movements; and
analyzing the first and second sets of features to determine whether they satisfy a validation condition corresponding to common decoded motor commands indicative of the individual's intended physical action resulting in the one or more movements;
d. said method comprises analyzing said control signal and selecting a corresponding physical action to be performed by an execution device;
e. said method comprises generating feedback data indicative of whether said condition is satisfied or not to be communicated to the individual;
f. the detected brain signals comprise EEG signals;
g. said method comprises preliminary analysis of the first measured data indicative of the brain signals, and upon identifying movement related signals in the first measured data, utilizing said movement related signals as a marker of voluntary movement onset, to select for analysis a part of the second measured data being collected from said voluntary movement onset;
h. said method comprises communicating data indicative of the individual's intended physical action to at least one of an execution device and an individual's assistant device;
i. said first and second measured data are provided from a storage system; and
j. said first and second measured data are concurrently provided from respective first and second measurement devices, said processing being performed in real time.

50. The method according to claim 49, wherein said processing comprises determining frequency and time evolution of the brain signals corresponding to the multiple sources distributed in the brain.

51. The method according to claim 50, wherein said processing comprises determining a time pattern of the motion signal being indicative of a motion type and quality.

52. The method according to claim 49, wherein said processing comprises applying machine learning analysis to the brain signals and the motion signals to define the respective first and second sets of features.

53. The method according to claim 52, wherein said processing comprises:
for each of the first and second data measured data, extracting from the respective measured data a plurality of features associated with motor commands, thereby generating first and second pluralities of features and utilizing machine learning results to assign first and second classification data to, respectively, the first set of features from said first plurality of features and the second set of features from said second plurality of features, thereby generating first and second classification data associated with the first and second measured data; and
determining whether the first and second classification data satisfy a condition of mutual validation of the motor command decoding obtained from the first and second measured data.

54. The method according to claim 53, wherein the second measured data comprises at least one time pattern of the motion signals sensed on at least one portion of the body, said extracting of the second set of features comprising analyzing said at least one time pattern and extracting at least kinematic landmarks to be included in the second set of features.

55. The method according to claim 54, wherein said extracting of the first set of features comprises identifying in the first measured data descriptive features specific to imaginary movements of said at least one portion of the body to be included in the first set of features.
